(19) United States
(12) Patent Application Publication
(10) Pub. No.: US 2009/0062714 A1
(43) Pub. Date: Mar. 5, 2009

(54) BANDAGES FOR USE ON TIPS OF FINGERS AND TOES

(76) Inventors: Jorge Mario Trujillo, Odessa, FL (US); Luz Maria Trujillo-Jaramillo, Odessa, FL (US)

Correspondence Address: Jorge Mario Trujillo, 69 Aqueduct Terrace, Odessa, FL, 33556, US

(21) Appl. No.: 11/848,704

(22) Filed: Aug. 31, 2007

(51) Int. Cl. A61F 13/10

(52) U.S. Cl. 602/54; 602/52; 602/55; 602/58

(57) ABSTRACT

A bandage is for use in covering a tip of a digit. The bandage includes a backing material and an absorbent material. The backing material includes a coupling mechanism, and is substantially planar. The absorbent material is coupled to the backing material and includes a first portion and a second portion. The first portion includes a first end, a second end, and a body extending therebetween. The body has a width that is narrower than a width of the digit, wherein the width of the digit is measured adjacent to the tip of the digit. When the bandage is applied to the digit, the first portion is configured to extend generally axially over a portion of the digit and the second portion is configured to extend at least partially circumferentially about a portion of the digit.

### BACKGROUND OF THE INVENTION

This invention relates generally to bandages, and more particularly, to bandages for use in covering and protecting the tips of fingers and/or toes of a person.

Adhesive bandages have long been used by consumers to cover a wound, such as an open cut, blister, or puncture of the skin. Generally, bandages shield the wound from exposure to dirt and containments that could cause injury to the wound or which may lead to infection. Moreover, bandages effectively isolate the wound from the surrounding atmosphere, absorb fluids from open wounds, help reduce the chance of scarring or infection, and promote the healing of the wound.

The most common adhesive bandages commercially available are generally rectangular in shape. More specifically, such bandages generally include an absorbent or padded portion that is secured against the wound with a pair of opposing adhesive portions extending outward from the absorbing portion. Such bandages work well on relatively flat body parts, but generally are not as effective for use on the tips of toes and/or fingers. For example, when a person has a wound on a finger tip, often a first bandage is secured to the person's finger tip with a first adhesive portion being secured between the person's fingernail and first knuckle, and the second adhesive portion being secured along the inner surface of the person's finger between the person's fingertip and palm. However, as is well known, when a flat bandage is applied over the tip of a finger or toe, excess material associated with the bandage tends to wrinkle and crimp, such that the absorbent portion may not effectively seal the wound from the surrounding environment. As such, germs and/or other contaminants may enter the wound area through openings and gaps defined in the wrinkled or crimped areas. Moreover, because of the excess material, such bandages may protrude awkwardly from the wearer's finger tip and/or limit the dexterity of the wearer's finger, are generally uncomfortable to the wearer, and generally have a tendency to fall off. As a result, it is common to wrap a second bandage or adhesive tape around the first bandage to ensure that the first bandage remains coupled to the finger. Furthermore, such bandages are generally difficult for a wearer to securely couple to the wound such that the absorbent portion is placed in an optimum position against the wound. The problems associated with such bandages may be even more compounded when used on the tip of a toe.

To facilitate protecting wounds to tips of fingers and toes, at least some known bandages are non-rectangular. For example, at least some known bandages are bowtie-shaped or “X”-shaped. Within such bandages, an absorbent portion is centrally located between two generally triangular-shaped adhesive portions. Although such bandages provide additional adhesive material that can be bonded to the skin or overlapped against other bandage portions, when such bandages are positioned such that the absorbent portion extends over the wound on the fingertip, the excess material of such bandages may still wrinkle and crimp. Moreover, such bandages are generally difficult to secure to the tip of a toe or finger such that the absorbent portion is positioned against the wound.

Other known bandages for use with the tips of fingers or toes use non-planar portions. For example, U.S. Pat. No 6,139,514 to Benson describes a finger bandage device that includes a tubular member having a closed distal end and an open proximal end. The tubular member is sized to fit snugly around an injured finger. Specifically, the injured finger is inserted into the tubular member and the bandage is secured tightly circumferentially about the entire finger. To limit creasing and wrinkling, such bandages are limited to being used with digits that are slightly smaller than the inner diameter of the tubular member. As a result, to accommodate different sized fingers/toes, consumers must maintain a large inventory of bandages of different sizes. Moreover, because the majority of the finger is inserted into the tubular member, finger dexterity is severely limited and patient discomfort may be increased by such a bandage. In addition, such bandages are more costly than flat bandages.

Similarly, U.S. Pat. No. 7,249,385 to Schukraft describes a protective apparatus for use with the tips of fingers or toes. The apparatus includes a semi-spherically shaped flexible member and an elongated tab that extends tangentially and radially about a circumferential edge of the flexible member. The injured finger/toe is inserted into the flexible member and the tab is wrapped about the digit to secure the apparatus to the digit. Moreover, because a person's digits are not generally semi-spherical, when such apparatus are coupled to the tip of a person's digits, generally at least a portion of the flexible member is creased, overlaps itself, and/or is wrinkled. To limit undesirable creasing and wrinkling, such bandages are limited to being used with digits that are sized slightly smaller than the inner diameter of the semi-spherical member. As a result, to accommodate different sized fingers/toes, consumers must maintain a large inventory of bandages of different sizes. Moreover, such bandages are more costly to fabricate than flat bandages.

### BRIEF SUMMARY OF THE INVENTION

In one aspect, a bandage for use in covering a tip of a digit is provided. The bandage includes a backing material and an absorbent material. The backing material includes a coupling mechanism, and is substantially planar. The absorbent material is coupled to the backing material and includes a first portion and a second portion. The first portion includes a first end, a second end, and a body extending therebetween. The body has a width that is narrower than a width of the digit, wherein the width of the digit is measured adjacent to the tip of the digit. When the bandage is applied to the digit, the first portion is configured to extend generally axially over a portion of the digit and the second portion is configured to extend at least partially circumferentially about a portion of the digit.

In another aspect, a bandage for use in covering a tip of a digit is provided. The bandage includes a backing material including an inner surface, an opposite outer surface, and a coupling mechanism. The backing material is substantially planar and has a first shape. The absorbent material is coupled to the backing material inner surface and has a second shape that is substantially similar to the backing material first shape. The absorbent material includes a first portion and a second portion. The first portion includes a first end, a second end, and a body extending therebetween. The body has a width that is narrower than a width of the digit as measured adjacent to the tip of the digit. When the bandage is applied to the digit, the first portion is configured to extend generally axially over a portion of the digit and said second portion is configured to extend at least partially circumferentially about a portion of the digit. The backing material coupling mechanism is configured to secure the bandage to the digit.

In a further aspect, a bandage for use in covering a tip of a digit is provided. The bandage includes a flexible backing material and an absorbent material. The backing material includes an inner surface, and an opposite outer surface. The inner surface includes an adhesive material applied thereto. The backing material is substantially planar and has a generally T-shaped cross-sectional profile defined by a first portion and a second portion. The flexible backing material is fabricated from a breathable material. The absorbent material is coupled to the backing material inner surface and has a generally T-shaped cross-sectional profile. The absorbent material includes a first portion and a second portion, wherein the first portion includes a first end, a second end, and a body extending therebetween. The body has a width that is narrower than a width of the digit as measured adjacent to the tip of the digit. When the bandage is applied to the digit, the absorbent material first portion is configured to extend generally axially over a portion of the digit and the absorbent material second portion is configured to extend at least partially circumferentially about a portion of the digit. The adhesive is configured to couple the bandage to the digit. The absorbent material is configured to absorb fluids.

### BRIEF DESCRIPTION OF THE DRAWINGS

FIG. 1 is a plan view of an exemplary bandage for use with a tip of a person's digit;

FIG. 2 is a perspective view of a person's finger in position to enable the bandage shown in FIG. 1 to be applied thereto;

FIG. 3 is a perspective view of the finger shown in FIG. 2 and with the bandage shown in FIG. 1 partially applied thereto; and

FIG. 4 is a perspective view of the finger shown in FIG. 2 and with the bandage shown in FIG. 1 being fully secured thereo.

### DETAILED DESCRIPTION OF THE INVENTION

As used herein, an element or step recited in the singular and proceeded with the word “a,” “an,” or “one” (and especially, “at least one”) should be understood as not excluding plural said elements or steps, unless such exclusion is explicitly stated. Furthermore, references to “one embodiment” (or to “other embodiments”) of the present invention are not intended to be interpreted as excluding either the existence of additional embodiments that also incorporate the recited features or of excluding other features described in conjunction with the present invention. Moreover, unless explicitly stated to the contrary, embodiments “comprising” or “having” an element or a plurality of elements having a particular property may include additional such elements not having that property.

In addition, as used herein and throughout, the term “wound” means and refers generally to a cut, an abrasion, a laceration, a bruise, a blister, a burn, a skin puncture, and/or any other similarly related injury to the tip of a person's digit. As used herein, the term “digit” generally refers to any one of multiple appendages or toes on a person's foot, including their hallux, or any of multiple digits on a person's hand, including their thumb.

Moreover, as used herein, the term “breathable” generally refers to a material that is substantially pervious to water vapors and gases. In other words, “breathable” barriers and “breathable” films allow water vapor to pass therethrough, but are substantially impervious to liquids, such as water.

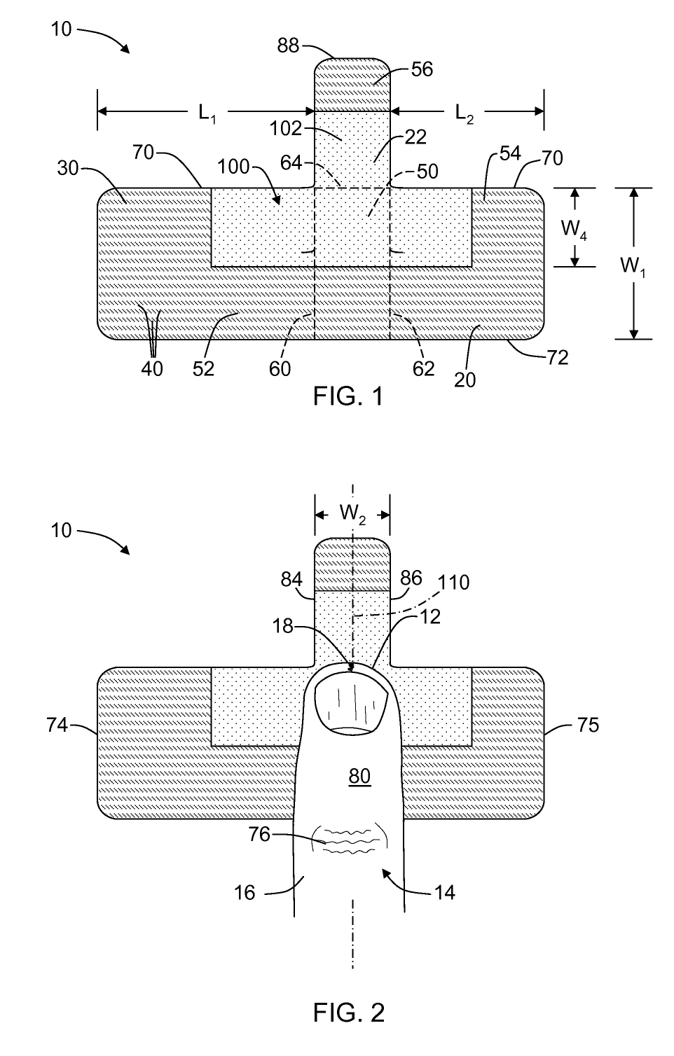

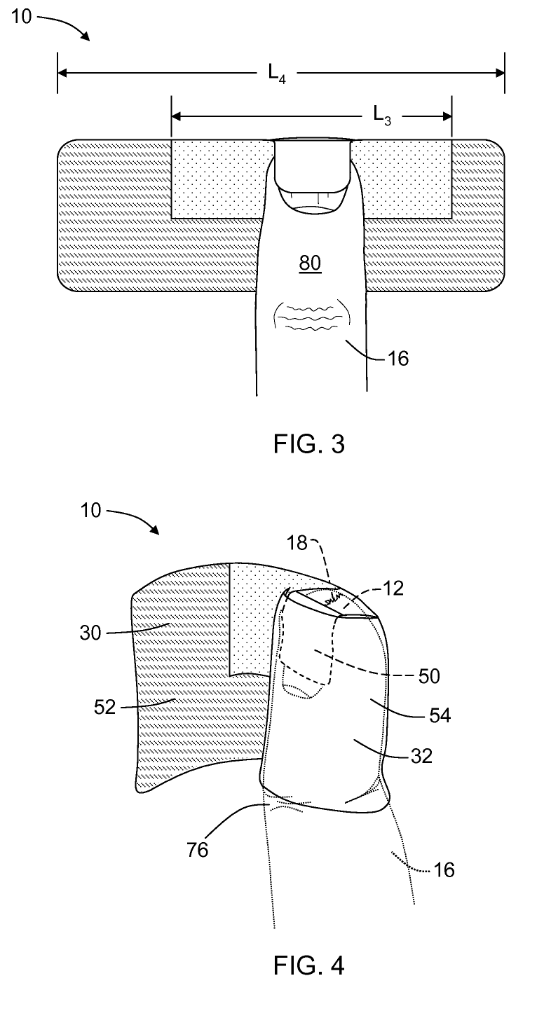

FIG. 1 is a plan view of an exemplary bandage 10 for use with a tip 12 of a person's digit 14, such as, but not limited to, a person's toe (not shown) or, as illustrated in the exemplary embodiment, a finger 16. FIG. 2 is a perspective view of a person's finger 16 in position to enable bandage 10 to be applied thereto. FIG. 3 is a perspective view of finger 16, and with bandage 10 partially applied thereto. FIG. 4 is a perspective view of finger 16 with bandage 10 being fully secured thereo. It should be noted that although bandage 10 is described herein as being applied to a digit 14 extending from a human, one of ordinary skill in the art should recognize that the description is not limiting and that alternatively, bandage 10 may be used for digits on animals as well, for example. Moreover, bandage 10 may also be used as a secondary wound dressing in which bandage 10 is used to hold a variety of different types of primary wound dressings, such as, but not limited to, sterile cotton gauze pads, on a wound 18 on a digit. Furthermore, bandage 10 may also be used to shield stitches on a digit from exposure to the surrounding atmosphere. As such, the description provided herein is only exemplary and should not be considered limiting.

In the exemplary embodiment, bandage 10 is disposable and is formed with a backing material 20 and an absorbent material 22 that is coupled to backing material 20. Alternatively, bandage 10 may be fabricated from materials that are washable to enable bandage 10 to be reused. More specifically, in the exemplary embodiment, backing material 20 is a one-piece member that is fabricated from a flexible material and that includes an inner surface 30 and an opposite outer surface 32. Inner surface 30 is substantially parallel to outer surface 32 and in the exemplary embodiment, inner and outer surfaces 30 and 32, respectively, are each substantially parallel. As such, bandage 10, when not in use, is substantially flat. Alternatively, to facilitate storage and/or ease of application, at least a portion of backing material 20 may be formed with a contour.

Backing material 20 is fabricated in the exemplary embodiment from a flexible and breathable material. Alternatively, depending on the intended use of bandage 10, backing material may be fabricated from a material that is pliable and/or is easily moldable. For example, in one embodiment, backing material 20 is fabricated from, but is not limited to being fabricated from, a cellulose material, cellulose acetate materials, poly(vinylidene chloride), nylon materials, and/or any combination thereof. In an alternative embodiment, backing material 20 may be fabricated from, but is not limited to being fabricated from, a synthetic material, a plastic material, a synthetic resin material, a non-absorbent polypropylene and/or any combination thereof Alternatively, backing material 20 may be fabricated from any material that enables bandage 10 to function as described herein.

In the exemplary embodiment, backing material 20 is perforated and includes a plurality of perforations or openings 40 that extend from backing material inner surface 30 to outer surface 32. Perforations enable air to pass through bandage 10 to facilitate healing of the wound 14. Alternatively, backing material 20 is formed without perforations 40 and is formed from any breathable material, such as a mesh material, that enables backing material 20 to function as described herein. In each embodiment, backing material 20 functions to retain topical ointments while substantially sealing the wound 18 from the surrounding environment. For example, in one embodiment, backing material 20 is fabricated from a gas permeable synthetic membrane that contains a colloid matrix.

As shown in FIG. 1, in the exemplary embodiment, backing material 20 has a seamless configuration and is generally T-shaped, as described in more detail below. Alternatively, backing material 20 may be formed with any shape, such as, but not limited to a lobed-shape, that enables bandage 10 to function as described herein. More specifically, in the exemplary embodiment, backing material 20 is formed integrally with a central portion 50, a pair of elongated lateral tabs 52 and 54, and an axial tab 56. In the exemplary embodiment, as described in more detail below, tabs 52 and 54 are substantially coaxially aligned with respect to each other and each extends outward from an opposite lateral side 60 and 62 of central portion 50. Moreover, in the exemplary embodiment, tab 56 is oriented substantially perpendicularly to tabs 52 and 54, and extends outward from an upper lateral side 64 of portion 50. Alternatively, any, or all, of tabs 52, 54, and /or 56 may extend obliquely from portion 50 or relative to the other tabs 52, 54, and/or 56.

Each tab 52 and 54 extends a respective length L₁ and L₂ outward in opposite directions from central portion 50. In the exemplary embodiment, length L₁ is longer than length L₂. Alternatively, lengths L₁ and L₂ may be any length, including having substantially the same length, that enables bandage 10 to function as described herein. In the exemplary embodiment, as described in more detail below, tab lengths L₁ and L₂ enable tabs 52 and 54 to extend at least partially circumferentially about the digit 14 when bandage 10 is applied to digit 14. For example, in one embodiment, tab lengths L₁ and L₂ enable tab 52 to partially overlap tab 54.

Moreover, in the exemplary embodiment, tabs 52 and 54 each have the same width W measured from an upper edge 70 of tabs 52 and 54, to a lower edge 72 of backing material 20. Edges 70 and 72 are connected together by a pair of opposite outer edges 74 and 75 that each extend between edges 70 and 72. In the exemplary embodiment, edges 70 and 72 are substantially linear and as such, width W is substantially uniform throughout tabs 50 and 52. Accordingly, in the exemplary embodiment, the portion of bandage 10 defined by tabs 52 and 54 and by central portion 50 is generally rectangular. Alternatively, tabs 52 and 54 may be formed with any shape, length, and/or width that enables bandage 10 to function as described herein. For example, in one alternative embodiment, at least one edge 70, 72, 74, and/or 75 is contoured, non-linear, and/or includes a recessed portion (not shown) that enables bandage 10 to avoid contact with a joint 76 connecting the distal and middle phalanges of the finger 16 to which the bandage 10 is applied.

In the exemplary embodiment, tab 56 extends outward from the upper lateral side 64 of bandage central portion 50 and has a width W₂ measured between a pair of opposing side edges 84 and 86. Sides 84 and 86 are connected together by an outer edge 88 that extends between sides 84 and 86. In the exemplary embodiment, side edges 84 and 86 are each substantially linear and are substantially parallel. As such, in the exemplary embodiment, width W₂ is substantially uniform throughout tab 56. Accordingly, in the exemplary embodiment, tab 56 has a generally rectangular shape. Alternatively, tab 56 may be formed with any shape, length, and/or width that enables bandage 10 to function as described herein. For example, in one alternative embodiment, at least one edge 84, 86, and/or 88 is contoured, non-linear, and/or includes a recessed portion (not shown).

In the exemplary embodiment, at least a portion of backing material inner surface 30 is coated at least partially with a known adhesive material (not shown). More specifically, in the exemplary embodiment, exposed areas of backing material 20 include an adhesive material that enables bandage 10 to be releasably secured to the person's skin 80 without casing any adverse reactions thereto. For example, in the exemplary embodiment, the adhesive material is formed as a coating that is applied to, or is formed integrally with, backing material 20. Alternatively, backing material 20 may include any material, such as, but not limited to, a pressure sensitive adhesive, that enables bandage 10 to be releasably secured to skin 80.

Alternatively, rather than an adhesive material, bandage 10, and more specifically, backing material 20, includes any known coupling mechanism that enables bandage 10 to be securely coupled in position to digit 14 such that bandage 10 may function as described herein. For example, bandage 10 may include, but is not limited to including a snap fitting mechanism, an interlocking device, hook and pile fasteners, hook and loop fasteners, tab and slot devices, tying systems, and/or clips.

In the exemplary embodiment, absorbent material 22 is fabricated from a sterile material that is formed in a pad-like configuration against backing material inner surface 30. More specifically, in the exemplary embodiment, material 22 is an occlusive, skin-comfortable material that can be, but is not limited to being, a non-porous film, an open-cell or closed cell foam, a woven or non-woven fabric, a laminate, or any combination thereof. Moreover, in the exemplary embodiment, absorbent material 22 is fabricated from, but is not limited to being fabricated from, a cloth fabric material, a gauze material, a linen material, a cotton material, any other natural material, a synthetic material, a cotton blends material, a silk material, woven paper, or any combination thereof.

Absorbent material 22 is coupled to inner surface 30 using any known coupling means, such as via a thermal bonding process, that enables bandage 10 to function as described herein. In the exemplary embodiment, absorbent material 22 is substantially centered with respect to central portion 50. More specifically, in the exemplary embodiment, absorbent material 22 is a one-piece member that includes a laterally-extending portion 100 and an axially-extending portion 102. Alternatively, bandage 10 may include a plurality of absorbent material portions. Accordingly, in the exemplary embodiment, absorbent material 22 is generally T-shaped. Moreover, in the exemplary embodiment, absorbent material 22 has a shape that is generally the same shape as that of backing material 20. Alternatively, absorbent material 22 may be formed with any shape, such as, but not limited to, a lobed-shape, that enables bandage 10 to function as described herein. For example, in an alternative embodiment, backing material 20 and absorbent material 22 each have different shapes.

More specifically, in the exemplary embodiment, absorbent material 22 is coupled to backing material 20 such that radially-extending portion 100 extends from a central portion 50 partially towards backing material outer edges 74 and 75. Accordingly, radially extending portion 100 has a length L₃ that is shorter than a length L₄ of backing material 20 as measured between edges 74 and 75. Furthermore, in the exemplary embodiment, radially extending portion 100 is coupled to backing material 20 such that portion 100 extends from backing material upper edges 70 partially towards backing material lower edge 72. Accordingly, in the exemplary embodiment, portion 100 has a width W₄ that is shorter than backing material width W₁. Alternatively, absorbent material 22 may be formed with any dimensions that enables bandage 10 to function as described herein.

In the exemplary embodiment, axially-extending portion 102 is formed integrally with radially-extending portion 100 and extends substantially perpendicularly outward from radially-extending portion 100 partially towards backing material outer edge 88. Alternatively, portion 102 may be formed with any dimensions that enables bandage 10 to function as described herein. Moreover, in an alternative embodiment, portion 102 may extend obliquely from portion 100. Furthermore, in the exemplary embodiment, portion 102 extends radially from backing material edge 84 to edge 86. Accordingly, in the exemplary embodiment, portion 102 is formed with approximately the same width as backing material tab 56 (i.e, width W₂). Moreover, in the exemplary embodiment, adhesive material is applied to the portion of inner surface 30 on tab 56 that is exposed between the absorbent material 22 and outer edge 88.

To facilitate comfort to the wearer, and to facilitate preventing an edge 70, 72, 74, or 75 from undesirably lifting or peeling from the wearer's digit 14 after bandage 10 has been applied, in the exemplary embodiment, the intersections defined between backing material edges 70 and 72 and side edges 74 and 75, as well as the intersections between tab side edges 84 and 86 and outer edge 88 are rounded. In an alternative embodiment, the intersections defined between tab edges 84 and 86 and backing material upper edge 70 are also rounded.

As is known, at least a pair of removable release sheets are provided to protect the adhesive material applied to backing material 20 and to maintain the sterility of the absorbent material 22 during storage and prior to bandage 10 being applied to wound 18. More specifically, in the exemplary embodiment, the exposed adhesive materials on the inner surface 30 of each tab 52, 54, and 56, are each covered with a removable sheet or shield material.

During use, when a person has a wound 18 at an outer tip 12 of a digit 14, such as finger 16, initially a bandage 10 is selected that includes a tab 56 that has a width W₂ that is slightly narrower than a width of digit 14, as measured at the tip 12 of the injured digit 14. In the exemplary embodiment, the shield material is peeled away from bandage 10 such that the adhesive material on backing material inner surface 30 is exposed. Digit 14 is then positioned against the inner surface 30 such that their digit 14 is generally centered radially with respect to tab 56 and is aligned generally coaxially to a centerline 110 extending through tab 56. Moreover, when positioned against bandage 10, the tip 12 of the digit 14 is positioned axially with respect to bandage 10 such that tip 12 extends generally along the upper lateral side 64 of bandage central portion 50.

After finger 16 is positioned against bandage inner surface 30, as described above, tab 56 is then rotated and is folded axially about tip 12. More specifically, as tab 56 is rotated about and over tip 12, absorbent material portion 102 is positioned against wound 18 and the outer edge 88 of tab 56 is positioned between tip 12 and the first knuckle 76 on the digit 14 to which bandage 10 is being applied. The adhesive material applied to tab 56 is then pressed against finger 16, such that absorbent material portion 102 is slightly compressed against the person's finger 16 and more specifically against wound 18. Because bandage 10 is flexible, as the absorbent material 22 is compressed against the person's finger 16, tab 56 essentially conforms against the person's finger 16, such that the absorbent material portion 102 substantially isolates the wound 18 from the surrounding environment. More specifically, in the exemplary embodiment, as tab 56 is coupled to an upper surface of finger 16, absorbent material portion 102 is compressed in a generally U-shape about tip 12 and against wound 18. Moreover, once positioned against wound 18, absorbent material 22 provides additional protection for wound 18, absorbs drainage from wound 18, and provides support to the tip 12 of the injured finger 16.

Bandage tab 54 is then rotated circumferentially about digit 14 such that tab 54 overlaps at least a portion of tab 56. More specifically, and as shown in FIG. 4, when tab 54 is rotated across about and around finger 16, the lower edge 72 of backing material 20 is either aligned with, or is between tip 12 and the joint 76 connecting the distal and middle phalanges of digit 14 to which bandage 10 is being applied. The adhesive material applied to tab 54 is then pressed against digit 14 and tab 56, such that tab 54 facilitates securing tab 56 to digit 16. More specifically, as tab 54 is pressed against tab 56, tab 56 is substantially sealed about wound 18 and against skin 80 at edges 84, 86, and 88.

Tab 52 is then rotated circumferentially about the person's finger 16 such that tab 52 a portion of tab 54 and, thus, also overlaps tab 56. More specifically, when tab 52 is rotated across about and around finger 16, again the lower edge 72 of backing material 20 is either aligned with, or is positioned between tip 12 and finger joint 76. The adhesive material applied to tab 52 is then pressed against an outer surface of tab 54, such that tab 52 facilitates securing bandage 10 to finger 16. Moreover, when tabs 54 and 56 are coupled to finger 16, absorbent material portion 100 extends more than circumferentially about finger 16 to facilitate providing comfort to the wearer and to ensure that any fluids that may leak from portion 102 are also absorbed. In addition, the circumferential positioning of portion 100 enables tabs 54 and 56 to more closely conform to the outer surface of finger 16.

Because the width W₂ of tab 56 is narrower than digit 14, and because absorbent material portions 100 and 102 enable tabs 52, 54, and 56 to more closely conform against the outer surface of finger 16, excess material that inevitably results when known flat bandages are fitted about multiple curved body parts, such as the tips of fingers and toes, is substantially eliminated. Moreover, because tab 54 overlaps tab 56, and because tab 52 overlaps tab 54, the bandage is facilitated to be securely coupled to finger 16 such that the outer edges, 84, 86, and 88 of tab 56 are overlapped by tabs 54 and/or tab 52. In addition, because bandage 10 conforms closely to the multi-curved surfaces of finger 16, ridges, folds, and protrusions inherent with conventional flat bandages are substantially eliminated. As such, the wound 18 is substantially sealed from the exterior environment along the entire periphery of tab 56. Furthermore, avenues of exposure for the introduction of germs and/or other contaminants to the wound 18 are also facilitated to be greatly inhibited, if not eliminated. Moreover, as may be appreciated more so by patients wearing such bandages 10 on their toes, pain, discomfort, and/or blistering associated with known bandages that include portions that protrude awkwardly outward from the tips of digits when coupled to the digit tips, are facilitated to be reduced, if not eliminated. In addition, because tab 54 overlaps tab 56, and because tab 52 overlaps tab 54, the bandage 10 is facilitated to be securely coupled to finger 16.

Furthermore, because bandage 10 is sized to ensure that backing material lower edge 72 and tab outer edge 88 are either aligned with, or are positioned between tip 12 and finger joint 76, bandage 10 is designed to facilitate reducing hindrance to the patient when performing operations requiring dexterity, such as typing. Moreover, as will be appreciated by one of ordinary skill in the art, the elimination of ridges, folds, and protrusions in bandage 10 facilitates enhancing the manual dexterity of the digit about which the protective apparatus is secured. In one embodiment, to assist a patient with the application process, bandage 10 may include indicia (not shown) that is used to identify, but is not limited to identifying, a size of the bandage (i.e. “large”), an intended digit for use with the bandage, (i.e. “for use with a ring finger”), and/or a sequence for applying the flaps to the digit (i.e., flap 1 should be folded first”). Similarly, in one embodiment, absorbent pad 22 may also include indicia indicating a desired location for the placement of a digit 14 during the application process.

The above-described bandage provides a patient with a dressing that includes an absorbent material that is coupled to a backing material. More specifically, the absorbent material is positioned to extend axially over the tip of the patient's finger and at least partially circumferentially about the tip of the patient's finger. In addition, the bandage includes a first tab that extends generally axially over the tip of the patient's finger and a pair of second tabs that extend at least partially circumferentially about the tip of the finger such that the first tab is overlapped fully by at least one of the second tabs. As such, the peripheral edges of the first tab are overlapped by the second tabs such that the wound is substantially sealed from the surrounding environment. As a result, a bandage is provided for use with the tips of digits that facilitates reducing the risks of contaminants entering a wound on a tip of the patient's digit, and that is more easily applied and secured in position to cover a wound than known bandages, without reducing the dexterity of the patient's digit, and without requiring the use of a secondary coupling means, such as a second bandage, to secure the bandage to the finger.

Exemplary embodiments of bandages are described above in detail. Although the bandages are herein described and illustrated in association with humans, it should be understood that the present invention may be used with non-humans as well. Moreover, it should also be noted that the components of each bandage are not limited to the specific embodiments described herein, but rather, aspects of each bandage and application method may be utilized independently and separately from other methods described herein.

While the invention has been described in terms of various specific embodiments, those skilled in the art will recognize that the invention can be practiced with modification within the spirit and scope of the claims.

What is claimed is:

1. A bandage for use in covering a tip of a digit, said bandage comprising:
a backing material comprising a coupling mechanism, said backing material is substantially planar; and
an absorbent material coupled to said backing material, said absorbent material comprising a first portion and a second portion, said first portion comprising a first end, a second end, and a body extending therebetween, said body having a width that is at least one of approximately the same width as and narrower than a width of the digit measured adjacent to the tip of the digit, when said bandage is applied to the digit, said first portion is configured to extend generally axially over a portion of the digit and said second portion is configured to extend at least partially circumferentially about a portion of the digit.

2. A bandage in accordance with claim 1 wherein said backing material comprises a first portion, a second portion, and a central portion extending between said first and second portions, said first portion is configured to extend generally axially over a portion of the digit to secure said absorbent material first portion to the digit, said second portion is configured to extend at least partially circumferentially about a portion of the digit to secure said absorbent material second portion to the digit.

3. A bandage in accordance with claim 2 wherein said backing material second portion is configured to overlap said backing material first portion when said bandage is fully applied to the digit.

4. A bandage in accordance with claim 2 wherein said backing material second portion comprises a first tab extending outward from a first side of said central portion and a second tab extending outward from a second side of said central portion.

5. A bandage in accordance with claim 4 wherein said second tab is configured to overlap at least a portion of said backing material first portion when said bandage is fully applied to the digit

6. A bandage in accordance with claim 4 wherein said first tab is configured to overlap at least a portion of said backing material first portion and at least a portion of said second tab when said bandage is fully applied to the digit.

7. A bandage in accordance with claim 1 wherein said backing material comprises an inner surface, an outer surface, and a plurality of perforations extending therebetween, said absorbent material is coupled against said inner surface, said backing material further comprises a coupling mechanism configured to secure said bandage to the digit.

8. A bandage in accordance with claim 1 wherein said backing material is flexible and is configured to substantially conform against an outer surface of the digit when said bandage is fully applied to the digit.

9. A bandage in accordance with claim 1 wherein said backing material is configured to compress said absorbent material first portion body substantially flush against an outer surface of the digit when said bandage is fully applied to the digit.

10. A bandage in accordance with claim 1 wherein said absorbent material is configured to substantially isolate a portion of the digit from a surrounding atmosphere when said bandage is fully applied to the digit.

11. A bandage for use in covering a tip of a digit, said bandage comprising:
a backing material comprising an inner surface, an opposite outer surface, and a coupling mechanism, said backing material is substantially planar and has a first shape; and
an absorbent material coupled to said backing material inner surface, said absorbent material having a second shape that is substantially similar to said backing material first shape, said absorbent material comprising a first portion and a second portion, said first portion comprising a first end, a second end, and a body extending therebetween, said body having a width that is narrower than a width of the digit as measured adjacent to the tip of the digit, when said bandage is applied to the digit, said first portion is configured to extend generally axially over a portion of the digit and said second portion is configured to extend at least partially circumferentially about a portion of the digit, said backing material coupling mechanism is configured to secure said bandage to the digit.

12. A bandage in accordance with claim 11 wherein said backing material comprises a first portion, a second portion, and a central portion extending between said first and second portions, said backing material first portion is configured to extend generally axially over a portion of the digit to secure said absorbent material first portion to the digit, said backing material second portion is configured to extend at least partially circumferentially about a portion of the digit to secure said absorbent material second portion to the digit.

13. A bandage in accordance with claim 12 wherein said absorbent material first portion is substantially centered with respect to said backing material first portion, said absorbent material second portion is coupled within said backing material second portion.

14. A bandage in accordance with claim 12 wherein said backing material second portion comprises a first tab having a first length and a second tab having a second length, said first tab extends outward from a first side of said central portion, said second tab extends outward from an opposite second side of said central portion, said backing material second portion is configured to overlap said backing material first portion when said bandage is fully applied to the digit.

15. A bandage in accordance with claim 12 wherein said backing material second portion comprises a first tab having a first length and a second tab having a second length, said first tab extends outward from a first side of said central portion, said second tab extends outward from an opposite second side of said central portion, said second tab is configured to overlap at least a portion of said first tab when said bandage is fully applied to the digit.

16. A bandage in accordance with claim 15 wherein said first tab first length is shorter than said second tab second length, said second tab is further configured to overlap at least a portion of said backing material first portion when said bandage is fully applied to the digit.

17. A bandage in accordance with claim 12 wherein said backing material second portion is further configured to substantially seal at least a portion of an outer periphery of said absorbent material first portion to the digit when said bandage is fully applied to the digit.

18. A bandage in accordance with claim 1 wherein said backing material is flexible and comprises a plurality of perforations extending between said inner and outer surfaces, said backing material is further configured to compress said absorbent material first portion body substantially flush against an outer surface of the digit when said bandage is fully applied to the digit.

19. A bandage for use in covering a tip of a digit, said bandage comprising:
a flexible backing material comprising an inner surface, and an opposite outer surface, said inner surface comprises an adhesive material applied thereto, said backing material is substantially planar and has a generally T-shaped cross-sectional profile defined by a first portion and a second portion, said flexible backing material is fabricated from a breathable material; and
an absorbent material coupled to said backing material inner surface, said absorbent material having a generally T-shaped cross-sectional profile and comprising a first portion and a second portion, said absorbent material first portion comprising a first end, a second end, and a body extending therebetween, said body having a width that is narrower than a width of the digit as measured adjacent to the tip of the digit, when said bandage is applied to the digit, said absorbent material first portion is configured to extend generally axially over a portion of the digit and said absorbent material second portion is configured to extend at least partially circumferentially about a portion of the digit, said adhesive is configured to couple said bandage to the digit, said absorbent material is configured to absorb fluids.

20. A bandage in accordance with claim 19 wherein said backing material comprises a first portion, a second portion, and a central portion extending between said first and second portions, said backing material first portion is configured to extend generally axially over a portion of the digit to secure said absorbent material first portion substantially flush against a portion of the digit, said backing material second portion comprises a first tab and a second tab that each extend outward from opposite sides of said second portion, said backing material second portion is configured to extend at least partially circumferentially about a portion of the digit such that at least a portion of said first tab is overlapped by said second tab to secure said absorbent material second portion to the digit.
